(19) United States
(12) Patent Application Publication
(10) Pub. No.: US 2024/0226514 A1
(43) Pub. Date: Jul. 11, 2024

(54) COMPOSITIONS COMPRISING A VAGINAL MOLD AND METHODS OF USE THEREOF

(71) Applicants: Duke University, Durham, NC (US); Mayo Foundation for Medical Education and Research, Rochester, MN (US)

(72) Inventors: Cassandra Kisby, Durham, NC (US); John Gebhart, Rochester, MN (US)

(21) Appl. No.: 18/405,331

(22) Filed: Jan. 5, 2024

Related U.S. Application Data
(60) Provisional application No. 63/437,536, filed on Jan. 6, 2023.

(51) Int. Cl. A61M 29/00

(52) U.S. Cl. CPC A61M 29/00; A61M 2207/10; A61M 2210/1475

(57) ABSTRACT

A vaginal mold with an improved shape and novel features. The vaginal mold is configured to be lightweight, inexpensive, comfortable, and compatible with vaginal secretions and other bodily functions. The improved shape allows the user to wear the device continuously for long periods while being easily insertable and removable.

### CROSS-REFERENCE TO RELATED APPLICATION

This application claims priority to U.S. Provisional Patent Application Ser. No. 63/437,536, filed Jan. 6, 2023, the contents of which are hereby incorporated by reference in their entirety.

### STATEMENT REGARDING FEDERALLY SPONSORED RESEARCH OR DEVELOPMENT

Not applicable.

### BACKGROUND OF THE INVENTION

### Field of the Invention (Technical Field)

The present invention relates generally to vaginal dilators. More particularly the present invention relates to vaginal molds, kits comprising vaginal molds, methodologies for the patient-specific custom manufacture of vaginal molds, and methodologies to treat patients with the embodiments of vaginal molds of the present invention.

### DESCRIPTION OF RELATED ART

Adequate vaginal length and girth are necessary for comfortable vaginal intercourse. However, certain medical conditions such as vaginal stenosis resulting from vulvovaginal dermatoses, radiation treatments (XRT) such as brachytherapy, atrophy from menopause or other hormonal changes, damage from physical trauma, and some surgical procedures (e.g., pelvic reconstructive surgery, vaginoplasty for vaginal agenesis, Mayer-Rokitansky-Küster-Hauser (MRKH) syndrome, or affirming care) may lead to vaginal stricture, agglutination of suture lines, and/or decrease in length, impacting sexual health and quality of life. For example, vaginal stenosis may cause burning, itching, spotting, frequent urinary tract infections and make sexual intercourse, use of tampons, or getting pelvic exams painful.

A mold for vaginal dilation can be used as primary prevention or treatment of vaginal stenosis, reducing the risk, or halting the progression of, concentric stricture, agglutination of suture lines, or loss of vaginal length. In fact, for neovaginal surgeries, a mold for vaginal dilation is necessary to the success of the surgery.

While there are some insertable molds currently available, all of these have drawbacks. Some are heavy and/or are designed to be handheld or used in a seated position, and thus the user must remain immobile while using the device or are limited by standardized length, girth, and shape (see e.g., FIGS. 1A and 1B prior art). They are also often difficult to place and remove. Others have a cylindrical shape that is prone to inconveniently sliding out of the user's body (see e.g., FIGS. 1B and 1C prior art). In addition, these devices lack fenestration, hindering the egress of vaginal excretions. Still other solutions are linked to negative side effects such as fistulas. This combination of factors contributes to decreased patient comfort and therefore compliance and increased complication rate.

Thus, there is an ongoing opportunity for improved vaginal molds.

### SUMMARY OF THE INVENTION

Disclosed herein is a vaginal mold comprising a rigid, at least partially hollow body defining a cavity and comprising a proximal end, a middle section, and a distal end, wherein the proximal end comprises a spherical dome-like, bulbous, globular shape, the middle section comprises tapering right and left sides, and the distal end comprises front and back tapering sides and bottlenecks to form a neck that ends into a knob comprising an opening to the cavity.

In one aspect, the vaginal mold further comprises one or more perforations to facilitate drainage of vaginal fluids through the opening of the cavity.

In one aspect, the one or more perforations of the vaginal mold are disposed on the proximal end of the body.

In one aspect, the proximal end of the mold has a rotationally symmetric spherical shape.

In one aspect, the vaginal mold further comprising a superior plateau aspect.

In one aspect, the knob of the vaginal mold further comprises a lip.

In one aspect, the body of the vaginal mold is made of a plastic resin.

In one aspect, the vaginal mold further comprising one of more internal channels to facilitate flow of fluid to the exterior surface of the mold that contacts a patient's vaginal tissue.

Disclosed herein is a kit comprising a plurality of the disclosed vaginal molds, wherein the molds are provided in incrementally larger sizes for gradual upsizing over time.

In one aspect the plurality comprises 2 or more vaginal molds.

In another aspect, the plurality comprises 3 or more vaginal molds and the kit further comprises at least one therapeutic agent, and a package insert with instructions for use.

Disclosed herein is a method of treating vaginal stenosis in a subject in need thereof using the vaginal mold of claim 1, the method comprising inserting the vaginal mold for a specified period of time to follow a treatment regimen.

In one aspect, the method further comprising administering to the subject an effective amount of one or more therapeutic agents.

In one aspect of the method, the vaginal mold can be inserted and removed with ease by the patient, and the regimen indicates gradual upsizing of incrementally larger vaginal mold sizes over time.

Disclosed herein is a method of fabricating the vaginal mold of claim 1, the method comprising obtaining one or more patient pelvic measurements; customizing the vaginal mold for the patient by generating a CAD 3D model of the vaginal mold from the patient measurements; and 3D printing the vaginal mold.

In one aspect, the method further comprising post-processing the 3D printed vaginal mold.

In another aspect of the method, the obtaining one or more patient pelvic measurements step comprises using imaging modalities (such as CT scanning and volumetric computed tomography imaging).

In another aspect of the method, the generating a CAD 3D model step comprises using one or more saved compatible templates.

In one aspect of the method, the post-processing the 3D printed vaginal mold step comprises smoothing the outer surface of the vaginal mold.

Disclosed herein is a vaginal mold comprising a rigid, at least partially hollow body defining a cavity and comprising a proximal end, a middle section, and a distal end, wherein the proximal end comprises a spherical rotationally symmetric, dome-like, bulbous, globular shape comprising one or more drainage perforations, wherein the middle section comprises tapering right and left sides, and wherein the distal end comprises front and back tapering sides and bottlenecks to form a neck that ends into a knob comprising a lip and an opening to the cavity.

These and other objects, features, and advantages of the present invention will become more readily apparent from the attached drawings and the detailed description of the preferred embodiments, which follow and may be attained by means of the instrumentalities and combinations particularly pointed out in the appended claim(s).

### BRIEF DESCRIPTION OF THE DRAWINGS

The preferred embodiments of the invention will hereinafter be described in conjunction with the appended drawings, which are incorporated into and form a part of the specification and together with the description, are provided to illustrate and not to limit the invention, where like designations denote like elements, and in which:

FIG. 1A shows commercially available sit-on vaginal dilators;

FIG. 1B shows commercially available hand-held dilators;

FIG. 1C shows a commercially available inflatable vaginal dilation stent;

FIG. 2 presents an isometric front view of an embodiment of a hollow vaginal mold of the present invention. Note that the rear view of the embodiment is identical;

FIG. 3A presents a bottom perspective isometric view of the embodiment of FIG. 1 showing side distal tapering on its midsection, front and back tapering, bottlenecking and an open knob at its distal end for removal and egress of vaginal secretions;

FIG. 3B presents a bottom view of the embodiment of FIG. 1. Through the opening of the distal knob, a perforation at the vaginal apex can be observed;

FIG. 3C presents a top view of the embodiment of FIG. 1, with the perforation to allow for vaginal secretion intake and subsequent egress through the opening on the knob;

FIG. 4 presents a side view of the embodiment of FIG. 1. Note that the right and left sides are identical mirror images of each other;

FIG. 5 presents a top side perspective of a different embodiment of a vaginal mold comprising a top plateau aspect;

FIG. 6 presents a cross-sectional front view of a different aspect of the invention in which a vaginal mold further comprises an internal channel and one or more perforations at the distal end for in situ administration of a therapeutic agent (e.g., vaginal estrogen cream) and/or irrigation for cleaning or flushing with, for example saline solutions;

FIG. 7 presents a cross-sectional front view of yet another aspect of the invention similar to the mold in FIG. 6, but comprising a channel expanding toward the proximal end and with more widely distributed exit holes for a divergent patter that widens the distribution of the therapeutic agent and/or flushing solution;

FIG. 8a presents a front view of a different embodiment of the invention with a more elongated proximal end;

FIG. 8b presents a side view of the embodiment of FIG. 8a;

FIG. 8c presents a perspective bottom view of the embodiment of FIG. 8a;

FIG. 9 presents a potential embodiment of orientation and support of the structure for 3D printing of an embodiment of the present invention;

FIG. 10 presents six potential sizes of embodiments of the invention for vaginal molds ranging from about 72 mm to about 92 mm in length. Note that the three vaginal molds on the bottom row comprise the superior plateau aspect shown in FIG. 5 and are thus a few millimeters shorter than their counterparts on the row above;

FIG. 11 depicts a flow chart for a method 400 of custom fabricating a patient-specific vaginal mold according to an embodiment of the invention;

FIG. 12 presents a flowchart for a clinical study; and

FIG. 13 presents a patient treatment workflow for treating patients for a one-year study.

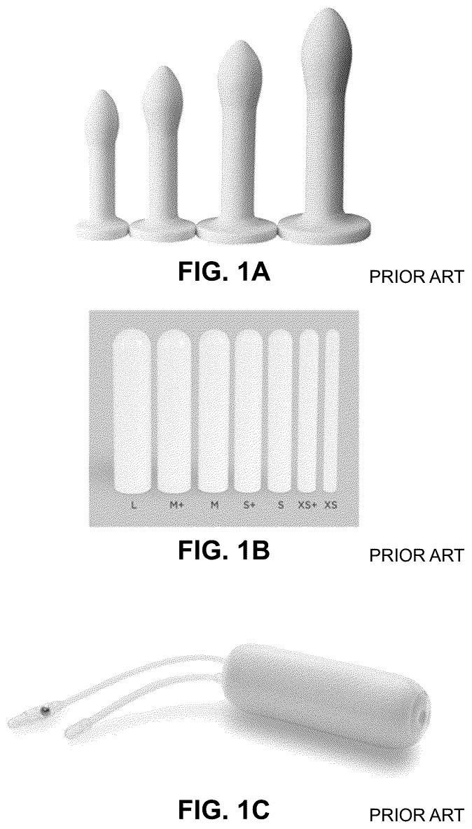

Like reference numerals refer to like parts throughout the several views of the drawings.

### DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the terms “about” or “approximately” are used interchangeably and mean a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of utilization, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, etc.). Thus, the subject of the herein disclosed methods can be a vertebrate, such as a mammal. Alternatively, the subject of the herein disclosed methods can be a human, non-human primate, horse, pig, rabbit, dog, sheep, goat, cow, cat, guinea pig, or rodent. The term does not denote a particular age or gender, and thus, adult and child subjects, whether biological female or transgender, are intended to be covered. In an aspect, a subject can be a human patient. In an aspect, a subject can suffer from vaginal stenosis from conditions such as vulvovaginal dermatoses, radiation treatments (XRT) such as brachytherapy, atrophy from menopause or other hormonal changes, damage from physical trauma and/or be in need of postsurgical care from surgical procedures such as pelvic reconstructive surgery, vaginoplasty for vaginal agenesis, Mayer-Rokitansky-Küster-Hauser (MRKH) syndrome, or affirming care, that results in or may lead to vaginal strictures, agglutination of suture lines, and/or decrease in length. In some embodiments, the subject comprises a human who is undergoing treatment using a device as prescribed herein.

As used herein, the term “diagnosed” means having been subjected to an examination by a person of skill, for example, a physician, and found to have a condition that can be diagnosed or treated by one or more of the disclosed vaginal molds or by one or more of the disclosed methods. For example, “diagnosed with a vaginal stricture condition” means having been subjected to an examination by a person of skill, for example, a physician, and found to have a condition that can be treated by one or more of the disclosed vaginal molds, or by one or more of the disclosed methods. For example, “suspected of having a vaginal stricture condition” can mean having been subjected to an examination by a person of skill, for example, a physician, and found to have a condition that can likely be treated by one or more of by one or more of the disclosed vaginal molds, or by one or more of the disclosed methods. In an aspect, an examination can be physical, can involve various tests (e.g., blood tests, genotyping, biopsies, etc.) and assays (e.g., enzymatic assay), or a combination thereof.

A “patient” refers to a subject afflicted with vaginal stenosis. In an aspect, a patient can refer to a subject that has been diagnosed with or is suspected of having vaginal stenosis. In an aspect, a patient can refer to a subject that has been diagnosed with or is suspected of having vaginal stenosis from a condition such as vulvovaginal dermatoses, radiation treatments (XRT), menopause atrophy, damage from physical trauma and/or in need of postsurgical care from surgical procedures such as pelvic reconstructive surgery, vaginoplasty for vaginal agenesis, Mayer-Rokitansky-Küster-Hauser (MRKH) syndrome, or affirming care, that results in or may lead to vaginal strictures, agglutination of suture lines, and/or decrease in length.

As used herein, the phrase “identified to be in need of treatment for a disorder,” or the like, refers to selection of a subject based upon need for treatment of the disorder. For example, a subject can be identified as having a need for treatment of a disorder (e.g., vaginal stenosis from a condition such as vulvovaginal dermatoses, radiation treatments (XRT), menopause atrophy, damage from physical trauma and/or in need postsurgical care from surgical procedures such as pelvic reconstructive surgery, vaginoplasty for vaginal agenesis, Mayer-Rokitansky-Küster-Hauser (MRKH) syndrome, or affirming care, that results in or may lead to vaginal strictures, agglutination of suture lines, and/or decrease in length). In an aspect, the identification can be performed by a person different from the person making the diagnosis. In an aspect, the administration can be performed by one who performed the diagnosis.

As used herein “vaginal stenosis” refers to the condition of atrophy, narrowing, tightening, shortening, closing, or loss of flexibility of the vaginal canal or vaginal opening resulting from, for example, scarring, dryness, or loss of mobility from vulvovaginal dermatoses, radiation treatments (XRT) such as brachytherapy, menopause or other hormone changes that may cause atrophy, damage from physical trauma, and some surgical procedures such as pelvic reconstructive surgery, vaginoplasty for vaginal agenesis, Mayer-Rokitansky-Küster-Hauser (MRKH) syndrome, or affirming care that results in or may lead to vaginal stricture, agglutination of suture lines, and/or decrease in length, impacting sexual health and quality of life.

As used herein, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject having a vagina that allows the subject to have vaginal intercourse without discomfort). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating vaginal stricture, agglutination of suture lines, and/or decrease in length can reduce the severity of an established vaginal medical condition in a subject by 1%-100% as compared to a control (such as, for example, an individual not having vaginal stenosis). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a vaginal stricture, agglutination of suture lines, and/or decrease in length. For example, treating a vaginal stricture, agglutination of suture lines, and/or decrease in length can reduce one or more symptoms of a vaginal condition in a subject by 1%-100% as compared to a control (such as, for example, an individual not having vaginal stenosis). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established vaginal medical condition. It is understood that treatment does not necessarily refer to a cure or complete ablation or eradication of a vaginal stricture, agglutination of suture lines, and/or decrease in length. However, in an aspect, treatment can refer to a cure or complete ablation or eradication of a vaginal stricture, agglutination of suture lines, and/or decrease in length.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a vaginal stricture, agglutination of suture lines, and/or decrease in length is intended. The words “prevent” and “preventing” and “prevention” also refer to prophylactic or preventative measures for protecting or precluding a subject (e.g., an individual) not having a given vaginal complication from progressing to that complication (such as, for example vaginal stricture, agglutination of suture lines, and/or decrease in length).

As used herein, the terms “administering” and “administration” refer to any method of providing one or more of the disclosed therapeutic agents thereof to a subject. Such methods are well-known to those skilled in the art and include, but are not limited to, the following: oral administration, transdermal administration, administration by inhalation, nasal administration, topical administration, in utero administration, intrahepatic administration, intravaginal administration, intracerebroventricular (ICV) administration, ophthalmic administration, intraaural administration, otic administration, intracerebral administration, rectal administration, sublingual administration, buccal administration, and parenteral administration, including injectable such as intravenous administration, intra-CSF administration, intra-cistern magna (ICM) administration, intra-arterial administration, intrathecal (ITH) administration, intramuscular administration, and subcutaneous administration. Administration can also include hepatic intra-arterial administration or administration through the hepatic portal vein (HPV). Administration of a disclosed therapeutic agent can comprise administration directly into the CNS or the PNS. Administration can be continuous or intermittent. Administration can comprise a combination of one or more route. In an aspect, a therapeutic agent, or any combination thereof can be concurrently and/or serially administered to a subject via multiple routes of administration. For example, in an aspect, administering a therapeutic agent, or any combination thereof can comprise intravenous administration and intra-cistern magna (ICM) administration. In an aspect, administering a disclosed therapeutic agent, or any combination thereof can comprise IV administration and intrathecal (ITH) administration. Various combinations of administration are known to the skilled person.

In an aspect, the skilled person can determine an efficacious dose, an efficacious schedule, and an efficacious route of administration for one or more of the disclosed therapeutic agents, or a combination thereof so as to treat or prevent a vaginal contracture, agglutination of suture lines, and/or decrease in length. In an aspect, the skilled person can also alter, change, or modify an aspect of an administering step to improve efficacy of one or more of the disclosed therapeutic agents, or a combination thereof.

As used herein, “modifying the method” can comprise modifying or changing one or more features or aspects of one or more steps of a disclosed method. For example, in an aspect, a method can be altered by changing the amount of one or more of the disclosed therapeutic agents, or a combination thereof administered to a subject, or by changing the frequency of administration of one or more of the disclosed therapeutic agents, or a combination thereof to a subject, or by changing the duration of time one or more of the disclosed therapeutic agents, or a combination are administered to a subject.

As used herein, “concurrently” means (1) simultaneously in time, or (2) at different times during the course of a common treatment schedule.

The term “contacting” as used herein refers to bringing one or more of the disclosed therapeutic agents, or a combination thereof together with a target area or intended target area in such a manner that the one or more of the disclosed therapeutic agents, or a combination thereof exert an effect on the intended target or targeted area either directly or indirectly. A target area or intended target area can be one or more of a subject's organs (e.g., uterus, vagina, etc.).

As used herein, “determining” can refer to measuring or ascertaining the presence and severity of a disease, such as, for example, vaginal stenosis. Methods and techniques used to determine the presence and/or severity of a vaginal stenosis are typically known to the medical arts. For example, the art is familiar with the ways to identify and/or diagnose the presence, severity, of vaginal stenosis.

As used herein, “effective amount” and “amount effective” can refer to an amount that is sufficient to achieve the desired result such as, for example, the treatment and/or prevention of a vaginal infection or a chronic pain condition. As used herein, the terms “effective amount” and “amount effective” can refer to an amount that is sufficient to achieve the desired an effect on an undesired condition (e.g., a vaginal infection). For example, a “therapeutically effective amount” refers to an amount that is sufficient to achieve the desired therapeutic result or to have an effect on undesired symptoms but is generally insufficient to cause adverse side effects. In an aspect, “therapeutically effective amount” means an amount of a disclosed therapeutic agent; that (i) treats the particular disease, condition, or (vaginal infections, dryness, pain, etc.), (ii) attenuates, ameliorates, or eliminates one or more symptoms of the particular disease, condition, or disorder e.g., vaginal infections, dryness, pain, etc.), or (iii) delays the onset of one or more symptoms of the particular disease, condition, or disorder described herein (e.g., vaginal infections, dryness, pain, etc.). The specific therapeutically effective dose level for any particular patient will depend upon a variety of factors including the disorder being treated and the severity of the disorder; the disclosed therapeutic agents employed; the disclosed methods employed; the age, body weight, general health, sex and diet of the patient; the time of administration; the route of administration; the rate of excretion of the therapeutic agents employed; the duration of the treatment; drugs used in combination or coincidental with other therapeutic agents, and other like factors well-known in the medical arts. For example, it is well within the skill of the art to start doses of therapeutic agents at levels lower than those required to achieve the desired therapeutic effect and to gradually increase the dosage until the desired effect is achieved. If desired, then the effective daily dose can be divided into multiple doses for purposes of administration. Consequently, a single dose of the disclosed therapeutic agents can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of therapeutic agents. In further various aspects, a preparation can be administered in a “prophylactically effective amount”; that is, an amount effective for prevention of a disease or condition, such as, for example, a vaginal infection, dryness, pain, etc.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., therapeutic agent, or a combination thereof) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., agent) to a subject having or diagnosed with a vaginal stricture, agglutination of suture lines, and/or decrease in length.

As used herein, “post-processing” and “3D printing post-processing” refer to any additional steps and procedures undertaken after the initial 3D printing phase of a device, and can include procedures such as smoothing out imperfections, sterilization to ensure aseptic conditions, and the like to make the device suitable for medical utilization by a subject, and to enhance the device's quality, functionality, and/or safety.

Unless otherwise defined, all technical terms used herein have the same meaning as commonly understood by one of ordinary skill in the art to which this disclosure belongs.

### B. Vaginal Molds

One aspect of the present disclosure provides a vaginal mold device. The device is configured to be lightweight, inexpensive, comfortable, and compatible with vaginal secretions and other bodily functions. The design as prescribed herein allows the user to wear the device continuously for long periods while being easily insertable and removable.

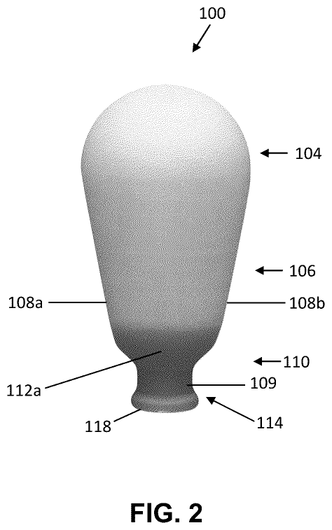

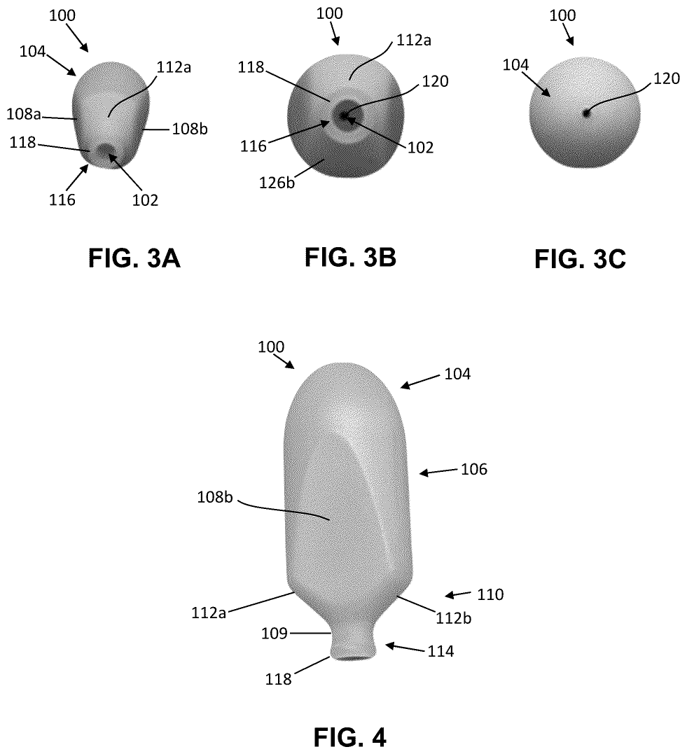

Referring now to FIGS. 2-4, in one non-limiting example embodiment, a vaginal mold 100 comprises an at least partly hollow body defining a cavity 102. In one embodiment, the vaginal mold 100 comprises a proximal end 104 comprising a spherical dome-like, bulbous, globular shape, similar to the top part of a balloon, or a lightbulb. In one aspect, the proximal end 104 has a rotationally symmetric spherical shape. This shape helps to prevent the vaginal mold 100 from slipping out during use. As can be best visualized in FIGS. 3A, and 3B and 4 (presenting a right-side view), the mold 100 comprises a middle section 106 comprising tapering right side 108a and left side 108b. Note that the right side and left side views of the mold 100 are identical, symmetric mirror images of each other.

In a preferred embodiment, the mold 100 comprises a distal end 110 comprising tapering front side 112a and tapering back side 112b. Note that a front view of the mold 100 is identical to a rear view. The distal end 110 then bottlenecks into a neck 109 that culminates into a knob 114 comprising an opening 116 to the cavity 102. In one aspect, the knob 114 provides an easy way for the patient to place and remove the mold (i.e., something to grip). In one embodiment, the knob 114 further comprises an optional lip 118. The tapering front side 112a and tapering back side 112b provide a narrowing at the area of the patient's urethra and colon/rectum to help prevent, for example, obstructed urination and defecation, and decrease the risk of fistulization. In one embodiment, the mold 100 further comprises one or more perforations 120 disposed, for example, at the axis of the distal end to facilitate drainage of vaginal fluids through the opening 116. In one aspect, the mold 100 improves discretion and is more comfortable to be worn for long periods of time while the patient is moving about.

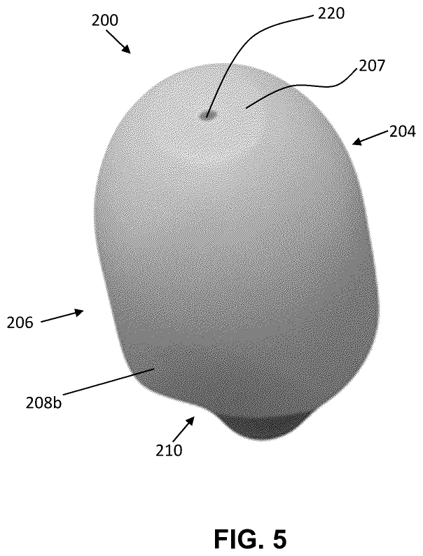

In a different embodiment, a vaginal mold 200 comprising an at least partly hollow body defining a cavity 202 and a similar spherical aspect of proximal end 204 than that of mold 100, further comprises a superior plateau aspect 207 (see FIG. 5 presenting a perspective top side view) to produce additional options for fitting and improve comfort of the mold. The vaginal mold 200, while maintaining the shape allows for slight shortening of the device if fine adjustments are needed. On the other hand, depending on the anatomy of the patient, there can be instances in which the plateau aspect 207 can reach all the way to the apex of the patient's vagina. In one embodiment the mold 200 also comprises a middle section 206 comprising tapering right side 208a and left side 208b.

In a preferred embodiment, the mold 200 also comprises a distal end 210 comprising tapering front side 212a and tapering back side 212b. The distal end 210 then bottlenecks into a neck 209 that culminates into a knob 214 comprising an opening 216 to a cavity 202. In one aspect, the knob 214 provides an easy way for the patient to place and remove the mold (i.e., something to grip). In one embodiment, the knob 14 further comprises an optional lip 218. The tapering front side 212a and tapering back side 212b provide a narrowing at the area of the patient's urethra and colon/rectum to help prevent, for example, obstructed urination and defecation, and decrease the risk of fistulization. In one embodiment, the mold 200 further comprises one or more perforations 220 disposed, for example, on the superior plateau aspect 207 of the distal end to facilitate drainage of vaginal fluids through the opening 216.

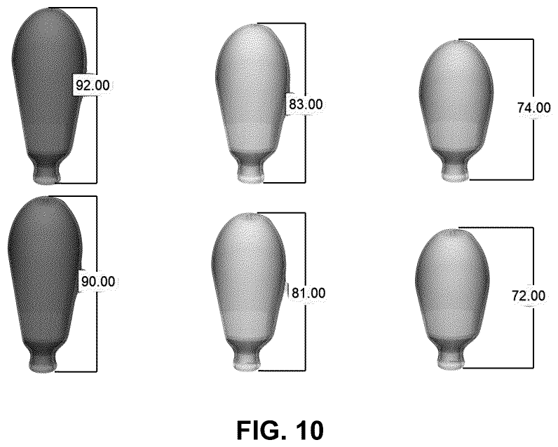

In one aspect, the vaginal molds are made in various sizes (e.g., 3 incrementally larger sizes) for gradual upsizing over time. See for example FIG. 10 presenting six potential sizes ranging from about 72 mm to about 92 mm in length. Note that the three vaginal molds on the bottom row comprise the superior plateau aspect 207 discussed above in reference to FIG. 5 and are thus a few millimeters shorter than their counterparts on the row above. In one aspect, the vaginal mold can range in length from about 20 mm (for example for pediatric cases) to about 140 mm; in another aspect the disclosed vaginal molds range in length from about 30 mm to about 110 mm; in another aspect the disclosed vaginal molds range in length from about 60 mm to about 100 mm; and in another aspect, the disclosed vaginal molds range in length from about 70 mm to about 95 mm.

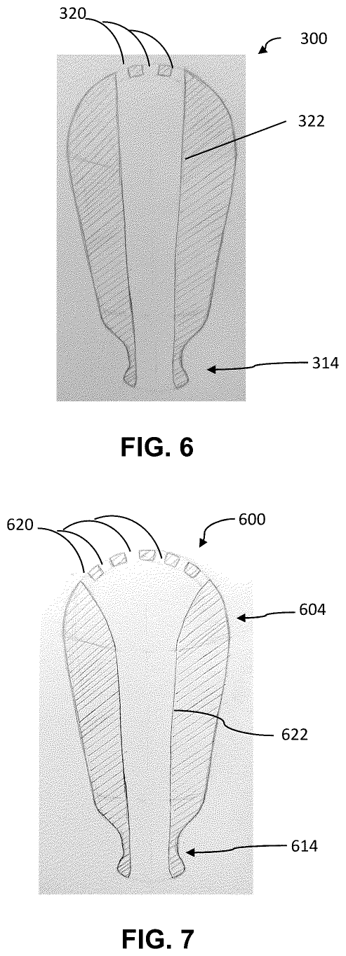

Referring to FIG. 6, in a different aspect of the invention, a vaginal mold 300 further comprises one or more internal channels 322 to allow for in situ administration of a therapeutic agent (e.g., vaginal estrogen cream, antibacterial/antifungal cream, anti-inflammatory/pain relief medication, vaginal moisturizer) and/or irrigation for cleaning or flushing with, for example saline solutions. In one aspect, an applicator, e.g., a syringe containing the therapeutic agent or flushing solution is attached to the knob 314 and when the therapeutic agent or flushing solution is injected into the internal channel(s) 322, it exits the device through one or more perforations 320. In one embodiment a plurality of channels are configured to direct for example, fluid to specific desired regions. In one aspect, a plurality of channel forms an internal network to direct a therapeutic agent to desired regions. Referring to FIG. 7, in a related aspect, a vaginal mold 600 comprises a central internal channel 622 that expands toward the proximal end 604. In one aspect the mold 600 comprises more widely distributed exit perforations 620 for a divergent patter that widens the distribution of the therapeutic agent and/or flushing solution as it exits the vaginal mold 600.

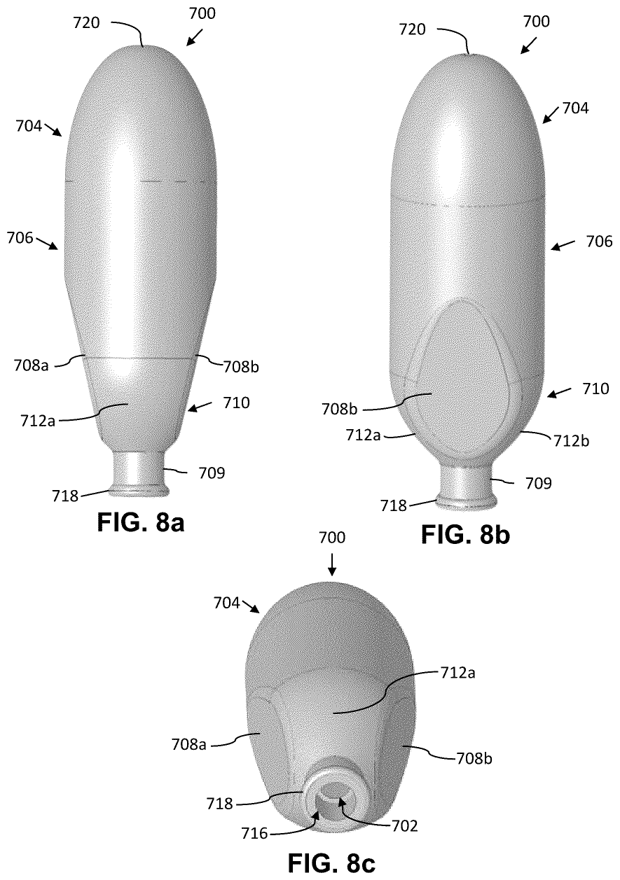

Referring now to FIGS. 8A-8C, in one embodiment, a vaginal mold 700 a proximal end 704 comprising a convex, less bulbous, and more elongated dome-like shape, resembling a capsule. In one aspect, the proximal end 704 has a rotationally symmetric spherical shape. In one aspect, while this shape helps to prevent the vaginal mold 700 from slipping out during use, it can also help in reaching all the way and dilating the apex of the vagina of the patient. The mold 700 comprises a middle section 706 comprising tapering right side 708a and left side 708b. Note that the right side and left side views of the mold 700 are identical, symmetric mirror images of each other.

In a preferred embodiment, the mold 700 comprises a distal end 710 comprising tapering front side 712a and tapering back side 712b. Note that a front view of the mold 700 is identical to a rear view. The distal end 710 then bottlenecks into a neck 709 that culminates into a knob 714 comprising an opening 716 to the cavity 702. In one aspect, the knob 714 provides an easy way for the patient to place and remove the mold (i.e., something to grip). In one embodiment, the knob 714 further comprises an optional lip 718. The tapering front side 712a and tapering back side 712b provide a narrowing at the area of the patient's urethra and colon/rectum to help prevent, for example, obstructed urination and defecation, and decrease the risk of fistulization. In one embodiment, the mold 700 further comprises one or more perforations 720 disposed, for example, at the axis of the distal end to facilitate drainage of vaginal fluids through the opening 716. In one aspect, the mold 700 improves discretion and is more comfortable to be worn for long periods of time while the patient is moving about.

In a different aspect, vaginal molds comprising one or more internal channels further comprise a superior plateau aspect in their proximal end, similar to the one discussed above in FIG. 5. In one embodiment, a superior plateau aspect is instrumental for in situ administration of a therapeutic agent (e.g., vaginal estrogen cream, antibacterial/antifungal cream, anti-inflammatory/pain relief medication, vaginal moisturizer) and/or irrigation for cleaning or flushing with, for example saline solutions.

### C. Methods of Fabrication

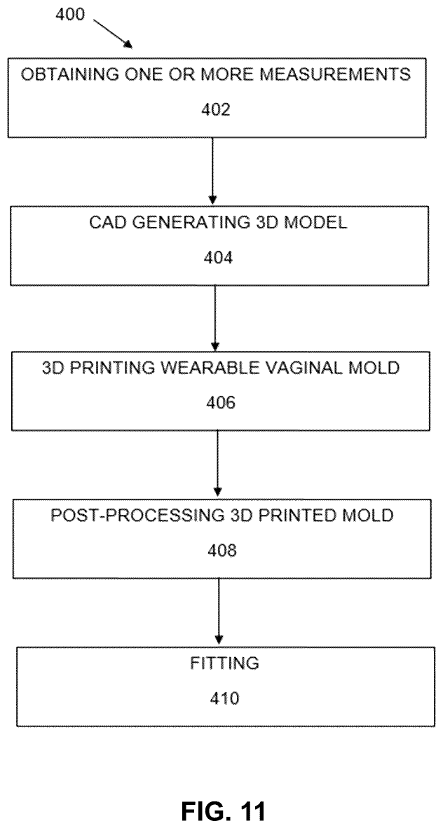

Referring to FIG. 11, in a different aspect of the invention, a method 400 of fabricating is used to make customized patient-specific vaginal molds. In one embodiment, the method 400 comprises step 402 obtaining measurements, for example, 3D anatomical measurements of, for example, a planned neovaginal space, or a unique anatomy (e.g., congenital anomalies like spina bifida or cloacal exstrophy, pediatric care, etc.). The measurements can be obtained, for example, from imaging modalities such as CT scanning and volumetric computed tomography imaging. In some instances, these dimensions can be further assessed visually by a healthcare practitioner, such as a surgeon. Alternatively, input from other sources can be used such as ultrasound, magnetic resonance, X-ray, and the like.

The method 400 further comprises step 404 for generating a 3D model of a vaginal mold based on the one or more measurements obtained in step 402.

The 3D model can be generated using computer aided design (CAD) software and can be manipulated by a healthcare provider to have a desired shape and/or dimensions. Examples of CAD software include, but are not limited to SolidWorks, AutoCAD, ZBrush, FreeCAD, Meshmixer, and SketchUp. In one embodiment, the mold design is created using a primary superior sphere, two cylinders (one larger main superior cylinder and a smaller inferior drain conduit cylinder), and a torus ring. Lofting is used to connect the main cylinder to the drain cylinder, and parallel oblique sketch planes from the tangent of the main cylinder are used to trim the anterior and posterior aspects of the model. The generated 3D models are then saved in a 3D printer-readable file format such as stereolithography (STL) or virtual reality modeling language (VRML).

In one aspect, the generating a CAD 3D model comprises using one or more saved compatible templates. Models previously generated for patients with similar medical conditions that are saved from previous projects can be used as a starting point for the custom, patient-specific generating a CAD 3D model step. In one aspect, the files are classified according to medical condition, demographics, and the like for identification. For example, a previously generated CAD 3D model for a pediatric patient may be a useful starting point for customizing a new vaginal mold for a similar patient.

In step 406 of the method 400 a 3D printer is used to fabricate the vaginal mold according to the model generated in step 404. There are many 3D printers commercially available from a variety of sources. One suitable option is the Formlabs Form 3B vat photopolymerization 3D printer (Formlabs, Boston, MA; FIG. 2B). The device can be made of any suitable rigid, washable, non-porous material, for example biocompatible plastics like the resins used to manufacture dentures. In Fused Deposition Modeling (FDM), a thermoplastic material is melted and laid on to the build platform in layer-by-layer fashion, until the object is formed. Suitable thermoplastics can include polylactic acid (PLA), acrylonitrile butadiene styrene (ABS), acrylonitrile styrene acrylate (ASA), nylon, polycarbonate (PC), PC-ABS blends, polyvinyl alcohol (PVA), thermoplastic elastomers (TPEs), high impact polystyrene (HIPS), and the like. Some 3D printers use stereolithography (SLA) where a UV laser beam selectively hardens the photo-polymer resin in layers. Each layer is solidified and built on top of next until the object is formed. Exemplary photopolymers for rigid medical devices are acrylic photopolymers such as RGD525, acrylonitrile butadiene styrene (ABS), polypropylene, rubber, and the like. In Selective Laser Sintering (SLS) a high-power laser beam fuses the powdered materials in layer-by-layer pattern to form the object. Materials suitable for this process are for example, nylon and polyamide.

In one embodiment, the method 400 further comprises step 408 for the post-processing 3D printed vaginal mold. In one aspect of the method, the parts are removed from build platforms and cleaned, e.g., soaked in 99.0% Isopropyl Alcohol for a suitable amount of time, for example, approximately 20 minutes. The supports are then detached from the molds and sanding is used to smooth the surface, for example, at the juncture between the vaginal mold and the printed supports. In one embodiment, a wet-sanding technique is employed to smooth the surface, with for example, a 600-grit paper to sand the entire surface until layer lines are smooth, followed by 2500-grit paper until audible scratching no longer occurs, and finally a 3M micro-fine pad is used in a dry-sanding technique to remove any remaining tactile deformities on the surface of the vaginal mold.

After the fabrication and post-processing steps are completed, a clinician/health practitioner can carry out the additional fitting step 410 to ensure that the mold fits the patient correctly, (i.e., “fitting” the vaginal mold with a clinician.), and potentially repeating some or all of the previous steps if the vaginal mold does not fit the patient correctly.

In a different aspect, the device can be formed using any suitable manufacturing method, for example, injection molding, casting, etc. and from a suitable material, for example biocompatible plastics, silicone, etc. In one embodiment, ready-made/off the shelf vaginal molds are fabricated by conventional methods, such as injection molding, machining, 3D printing, etc., in various optional sizes.

### D. Methods of Treating and/or Preventing Vaginal Stenosis, and/or Vaginal Stenosis Disease Progression

The embodiments of the present invention further include methods of treating and/or preventing vaginal stenosis, and/or halting vaginal stenosis progression in a subject in need thereof with the above-described embodiments of vaginal molds. In one aspect, the preventing vaginal stenosis, and/or halting vaginal stenosis progression in a subject in need thereof is carried out utilizing the above-described embodiments of vaginal molds in combination with one or more therapeutic agents. In one aspect the disclosed vaginal molds are used by the patient continuously for long periods as applicable through a clinician's designed treatment regimen or protocol.

In one aspect, a treatment regimen comprises, for example, disinfecting/cleaning a disclosed vaginal mold, lubricating the disclosed vaginal mold, inserting the mold in the subject's vagina for a determined daily cumulative time (e.g., 1 minute, 15 minutes, 30 minutes, 45 minutes, 1 hour, 2 hours, 3 hours, 4 hours, 5 hours, 6 hours, 7 hours, 8 hours, 10 hours, 12 hours, 16, hours, 18, hours, 20, hours, 22, hours, 24 hours), for a sufficiently long span of time to treat and/or prevent vaginal stenosis, and/or halting vaginal stenosis progression in a subject (e.g., 1 week, 2 weeks, 3 weeks, 4 weeks, 5 weeks, 6 weeks, 8 weeks, 8 weeks, 9 weeks, 10 weeks, 11 weeks, 12 weeks, 14 weeks, 16 weeks, 18 weeks, 20 weeks, 22 weeks, 24 weeks, 26, weeks, 28 weeks, 30 weeks, 32 weeks, 34 weeks, 36 weeks, 38 weeks, 40 weeks, 42 weeks, 44 weeks, 48 weeks, 50 weeks, 52 weeks, or longer). In one aspect, the clinician adjusts the daily time and span of treatment regimen based on, for example, the subject's reported pain level during vaginal intercourse, the flexibility of the subject's vagina, the girth, or the length of the subject's vagina, and the like.

Disclosed herein is a method of treating and/or preventing vaginal stenosis, and/or halting vaginal stenosis progression in a subject in need thereof comprising utilizing a disclosed vaginal mold for a suitable length of time daily and administering a therapeutically effective amount of a disclosed therapeutic agent for treating and/or preventing or halting the progression of a co-morbidity.

Disclosed herein is a method of treating and/or preventing vaginal stenosis, and/or halting vaginal stenosis progression in a subject in need thereof comprising utilizing a disclosed vaginal mold continuously for a suitable length of time daily and administering a therapeutically effective amount of a combination of disclosed therapeutic agents for treating and/or preventing or halting the progression of one or more co-morbidities.

In one aspect, a suitable length of time for daily use of a disclosed vaginal molds by the patient is for between approximately 1 week and approximately 75 weeks or more. In one aspect, the disclosed vaginal molds are used daily by the patient for between approximately 2 weeks and approximately 52 weeks. In one aspect, the disclosed vaginal molds are used by the patient for between approximately 6 weeks and approximately 40 weeks. In one aspect, the disclosed vaginal molds are used by the patient for between approximately 12 weeks and approximately 26 weeks.

Disclosed herein is a method of treating and/or preventing vaginal stenosis, and/or halting vaginal stenosis progression in a subject in need thereof comprising sanitizing a disclosed vaginal mold, inserting the disclosed vaginal mold in the subject's vagina, and implementing a treatment regimen comprising wearing the vaginal mold for between about 3 and about 23.5 cumulative hours a day for between about 2 weeks and about 52 weeks.

In one aspect, the disclosed vaginal mold is worn every other day. In one aspect, the disclosed vaginal mold is worn overnight. In one aspect, the disclosed vaginal mold is worn during the day while the subject is moving about.

Disclosed herein is a method of treating and/or preventing vaginal stenosis, and/or halting vaginal stenosis progression in a subject in need thereof comprising inserting a disclosed vaginal mold in the subject's vagina and implementing a treatment regimen comprising wearing the disclosed vaginal mold for between about 0.25 and about 12 cumulative hours a day for between about 2 weeks and about 52 weeks and administering an effective amount of at least one therapeutic agent to the patient.

Disclosed herein is a method of treating and/or preventing vaginal stenosis, and/or halting vaginal stenosis progression in a subject in need thereof comprising inserting a disclosed vaginal mold in the subject's vagina and implementing a treatment regimen comprising wearing the disclosed vaginal mold for between bout 0.1 hours and about 24 hours a day for between about 2 weeks and about 78 weeks, and administering an effective amount of a combination of two or more therapeutic agents to the patient.

Disclosed herein is a method of treating and/or preventing vaginal stenosis, and/or halting vaginal stenosis progression in a subject in need thereof comprising sanitizing a disclosed vaginal mold, inserting the disclosed vaginal mold in the subject's vagina, and implementing a treatment regimen comprising wearing the vaginal mold for between about 3 and about 23.5 cumulative hours a day for between about 2 weeks and about 52 weeks, and further monitoring the patient's response.

Disclosed herein is a method of treating and/or preventing vaginal stenosis, and/or halting vaginal stenosis progression in a subject in need thereof comprising disinfecting and lubricating a disclosed vaginal mold, inserting the sterile and lubricated disclosed vaginal mold in the subject's vagina, implementing a treatment regimen for the subject comprising wearing the vaginal mold for between 3 hours and about 23.5 hours a day for between about 2 weeks and about 52 weeks, washing the vaginal mold daily, and administering an effective amount of at least one therapeutic agent to the patient, monitoring the patient's response, and adapting the treatment regimen based on the subjects response to the treatment regimen.

In one aspect, the patient reported results affect the clinician's response and adaptation of the treatment regimen. In one aspect, when the patient reports discomfort when using the mold, the clinician further evaluates to optimize the size of the disclosed vaginal mold(s) and/or adjusts the time of the treatment regimen. In one aspect, if the subject reports any discomfort when using the mold, the method further comprises refitting, obtaining additional measurements or remeasuring, adjusting, or manufacturing a new vaginal mold, and updating the time of treatment regimen.

In one aspect, the regimen indicates gradually upsizing with incrementally larger vaginal mold sizes over time. In one aspect, the incrementally larger vaginal molds comprise a plurality of molds. In one aspect, the plurality comprises 3 incrementally larger molds. In one aspect, the plurality comprises at least two incrementally larger molds. In one aspect the plurality comprises four or more incrementally larger molds. In one aspect, the plurality comprises six or more incrementally larger vaginal molds.

As used herein, the term “subject” refers to the target of utilization, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal. Alternatively, the subject of the herein disclosed methods can be a human, non-human primate, horse, pig, rabbit, dog, sheep, goat, cow, cat, guinea pig, or rodent. The term does not denote a particular age or gender, and thus, adult and child subjects, whether biological female or transgender, are intended to be covered. In an aspect, a subject can be a human patient. In an aspect, a subject can suffer from vaginal stenosis from conditions such as vulvovaginal dermatoses, radiation treatments (XRT) such as brachytherapy, menopause atrophy, damage from physical trauma and/or be in need of postsurgical care from surgical procedures such as pelvic reconstructive surgery, vaginoplasty for vaginal agenesis, Mayer-Rokitansky-Küster-Hauser (MRKH) syndrome, or affirming care, that results in or may lead to vaginal strictures, agglutination of suture lines, and/or decrease in length. In some embodiments, the subject comprises a human who is undergoing treatment using a device as prescribed herein. In one aspect the subject is a patient undergoing neovagina surgery for gender affirming care (i.e., is a transgender woman), or because the patient was born without a vagina.

In a different aspect, the disclosed vaginal mold further comprises one or more internal channels to allow for in situ administration of a therapeutic agent.

In one aspect the disclosed methods improve patient compliance because the disclosed vaginal molds are comfortable to wear for long periods of time and are easily insertable and removable. In one aspect, the disclosed method improves patient compliance because the patient is capable of inserting and removing the disclosed vaginal molds with ease and without discomfort or pain.

In a different aspect the disclosed methods reduce the patient's number of doctor's visits because the patient is capable of cleaning the disclosed vaginal molds and inserting them or removing them with ease. In one aspect the disclosed methods reduce the patient's embarrassment.

Methods and techniques to monitor a subject's response to a disclosed method can comprise qualitative (or subjective) means as well as quantitative (or objective) means. In an aspect, qualitative means (or subjective means) can comprise a subject's own perspective. For example, a subject can report how he/she is feeling, whether he/she has experienced improvements and/or setbacks, whether he/she has experienced an amelioration or an intensification of one or more symptoms, or a combination thereof. In an aspect, quantitative means (or objective means) can comprise methods and techniques that include, but are not limited to, the following: (i) fluid analysis (e.g., tests of a subject's fluids including but not limited to aqueous humor and vitreous humor, bile, blood, blood serum, breast milk, cerebrospinal fluid, cerumen (earwax), digestive fluids, endolymph and perilymph, female ejaculate, gastric juice, mucus (including nasal drainage and phlegm), peritoneal fluid, pleural fluid, saliva, sebum (skin oil), semen, sweat, synovial fluid, tears, vaginal secretion, vomit, and urine), (ii) imaging (e.g., ordinary x-rays, ultrasonography, radioisotope (nuclear) scanning, computed tomography (CT), magnetic resonance imaging (MRI), positron emission tomography (PET), and angiography), (iii) endoscopy (e.g., laryngoscopy, bronchoscopy, esophagoscopy, gastroscopy, GI endoscopy, coloscopy, cystoscopy, hysteroscopy, arthroscopy, laparoscopy, mediastinoscopy, and thoracoscopy), (iv) analysis of organ activity (e.g., electrocardiogramactroencephalography (EEG), and pulse oximetry), (v) biopsy (e.g., removal of tissue samples for microscopic evaluation), and (vi) genetic testing.

### E. Agents

### Biologically Active Agents

As used herein, the term “biologically active agent” or “biologic active agent” or “bioactive agent” means an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents. It is understood that a biologically active agent can be used in connection with administration to various subjects, for example, to humans (i.e., medical administration) or to animals (i.e., veterinary administration). As used herein, the recitation of a biologically active agent inherently encompasses the pharmaceutically acceptable salts thereof.

### Pharmaceutically Active Agents

As used herein, the term “pharmaceutically active agent” includes a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA). Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a radiosensitizer, the combination of a radiosensitizer and a chemotherapeutic, a steroid, a xanthine, anti-inflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, timol hemihydrate, levobunolol hydrochloride, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists (i.e., alpha adrenergic receptor agonist) such as clonidine, brimonidine tartrate, and apraclonidine hydrochloride; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; prostaglandin analogs such as latanoprost, travoprost, and bimatoprost; cholinergics (i.e., acetylcholine receptor agonists) such as pilocarpine hydrochloride and carbachol; anti-Vascular endothelial growth factor (VEGF) aptamers such as pegaptanib; anti-VEGF antibodies (including but not limited to anti-VEGF-A antibodies) such as ranibizumab and bevacizumab; carbonic anhydrase inhibitors such as methazolamide, brinzolamide, dorzolamide hydrochloride, and acetazolamide; antiarrhythmic agents such as quinidine, lidocaine, tocainide hydrochloride, mexiletine hydrochloride, digoxin, verapamil hydrochloride, propafenone hydrochloride, flecaimide acetate, procainamide hydrochloride, moricizine hydrochloride, and diisopyramide phosphate; antiparkinsonian agents, such as dopamine, L-Dopa/Carbidopa, selegiline, dihydroergocryptine, pergolide, lisuride, apomorphine, and bromocryptine; anticoagulant and antiplatelet agents such as coumadin, warfarin, acetylsalicylic acid, and ticlopidine; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides; probiotic microorganisms; and prebiotics substances. It is understood that a pharmaceutically active agent can be used in connection with administration to various subjects, for example, to humans (i.e., medical administration) or to animals (i.e., veterinary administration). As used herein, the recitation of a pharmaceutically active agent inherently encompasses the pharmaceutically acceptable salts thereof.

### Anti-Bacterial Agents

As used herein, anti-bacterial agents are known to the art. For example, the art generally recognizes several categories of anti-bacterial agents including (1) penicillins, (2) cephalosporins, (3) quinolones, (4) aminoglycosides, (5) monobactams, (6) carbapenems, (7) macrolides, and (8) other agents. For example, as used herein, an anti-bacterial agent can comprise Afenide, Amikacin, Amoxicillin, Ampicillin, Arsphenamine, Augmentin, Azithromycin, Azlocillin, Aztreonam, Bacampicillin, Bacitracin, Balofloxacin, Besifloxacin, Capreomycin, Carbacephem (loracarbef), Carbenicillin, Cefacetrile (cephacetrile), Cefaclomezine, Cefaclor, Cefadroxil (cefadroxyl), Cefalexin (cephalexin), Cefaloglycin (cephaloglycin), Cefalonium (cephalonium), Cefaloram, Cefaloridine (cephaloradine), Cefalotin (cephalothin), Cefamandole, Cefaparole, Cefapirin (cephapirin), Cefatrizine, Cefazaflur, Cefazedone, Cefazolin (cephazolin), Cefcanel, Cefcapene, Cefclidine, Cefdaloxime, Cefdinir, Cefditoren, Cefedrolor, Cefempidone, Cefepime, Cefetamet, Cefetrizole, Cefivitril, Cefixime, Cefluprenam, Cefmatilen, Cefmenoxime, Cefmepidium, Cefmetazole, Cefodizime, Cefonicid, Cefoperazone, Cefoselis, Cefotaxime, Cefotetan, Cefovecin, Cefoxazole, Cefoxitin, Cefozopran, Cefpimizole, Cefpirome, Cefpodoxime, Cefprozil (cefproxil), Cefquinome, Cefradine (cephradine), Cefrotil, Cefroxadine, Cefsumide, Ceftaroline, Ceftazidime, Ceftazidime/Avibactam, Cefteram, Ceftezole, Ceftibuten, Ceftiofur, Ceftiolene, Ceftioxide, Ceftizoxime, Ceftobiprole, Ceftriaxone, Cefuracetime, Cefuroxime, Cefuzonam, Cephalexin, Chloramphenicol, Chlorhexidine, Ciprofloxacin, Clarithromycin, Clavulanic Acid, Clinafloxacin, Clindamycin, Cloxacillin, Colimycin, Colistimethate, Colistin, Crysticillin, Cycloserine 2, Demeclocycline, Dicloxacillin, Dirithromycin, Doripenem, Doxycycline, Efprozil, Enoxacin, Ertapenem, Erythromycin, Ethambutol, Flucloxacillin, Flumequine, Fosfomycin, Furazolidone, Gatifloxacin, Geldanamycin, Gemifloxacin, Gentamicin, Glycopeptides, Grepafloxacin, Herbimycin, Imipenem, Isoniazid, Kanamycin, Levofloxacin, Lincomycin, Linezolid, Lipoglycopeptides, Lomefloxacin, Meropenem, Meticillin, Metronidazole, Mezlocillin, Minocycline, Mitomycin, Moxifloxacin, Mupirocin, Nadifloxacin, Nafcillin, Nalidixic Acid, Neomycin, Netilmicin, Nitrofurantoin, Norfloxacin, Ofloxacin, Oxacillin, Oxazolidinones, Oxolinic Acid, Oxytetracycline, Oxytetracycline, Paromomycin, Pazufloxacin, Pefloxacin, Penicillin G, Penicillin V, Pipemidic Acid, Piperacillin, Piromidic Acid, Pivampicillin, Pivmecillinam, Platensimycin, Polymyxin B, Pristinamycin, Prontosil, Prulifloxacin, Pvampicillin, Pyrazinamide, Quinupristin/dalfopristin, Rifabutin, Rifalazil, Rifampin, Rifamycin, Rifapentine, Rosoxacin, Roxithromycin, Rufloxacin, Sitafloxacin, Sparfloxacin, Spectinomycin, Spiramycin, Streptomycin, Sulbactam, Sulfacetamide, Sulfamethizole, Sulfamethoxazole, Sulfanilimide, Sulfisoxazole, Sulphonamides, Sultamicillin, Teicoplanin, Telavancin, Telithromycin, Temafloxacin, Tetracycline, Thiamphenicol, Ticarcillin, Tigecycline, Tinidazole, Tobramycin, Tosufloxacin, Trimethoprim, Trimethoprim-Sulfamethoxazole, Troleandomycin, Trovafloxacin, Tuberactinomycin, Vancomycin, Viomycin, or pharmaceutically acceptable salts thereof (e.g., such as, for example, chloride, bromide, iodide, and periodate), or a combination thereof. As used herein, the recitation of an anti-bacterial agent inherently encompasses the pharmaceutically acceptable salts thereof.

### Anti-Fungal Agents

Anti-fungal agents are known to the art. The art generally recognizes several categories of anti-fungal agents including (1) azoles (imidazoles), (2) antimetabolites, (3) allylamines, (4) morpholine, (5) glucan synthesis inhibitors (echinocandins), (6) polyenes, (7) benoxaaborale; (8) other antifungal/onychomycosis agents, and (9) new classes of antifungal/onychomycosis agents. For example, as used herein, an anti-fungal agent can comprise Abafungin, Albaconazole, Amorolfin, Amphotericin B, Anidulafungin, Bifonazole, Butenafine, Butoconazole, Candicidin, Caspofungin, Ciclopirox, Clotrimazole, Econazole, Fenticonazole, Filipin, Fluconazole, Flucytosine, Griseofulvin, Haloprogin, Hamycin, Isavuconazole, Isoconazole, Itraconazole, Ketoconazole, Micafungin, Miconazole, Naftifine, Natamycin, Nystatin, Omoconazole, Oxiconazole, Polygodial, Posaconazole, Ravuconazole, Rimocidin, Sertaconazole, Sulconazole, Terbinafine, Terconazole, Tioconazole, Tolnaftate, Undecylenic Acid, Voriconazole, or pharmaceutically acceptable salts thereof, or a combination thereof. In an aspect, an anti-fungal agent can be an azole. Azoles include, but are not limited to, the following: clotrimazole, econazole, fluconazole, itraconazole, ketoconazole, miconazole, oxiconazole, sulconazole, and voriconazole. As used herein, the recitation of an anti-fungal agent inherently encompasses the pharmaceutically acceptable salts thereof.

### Anti-Viral Agents

Anti-viral agents are known to the art. As used herein, for example, an anti-viral can comprise Abacavir, Acyclovir (Aciclovir), Adefovir, Amantadine, Ampligen, Amprenavir (Agenerase), Umifenovir (Arbidol), Atazanavir, Atripla, Baloxavir marboxil (Xofluza), Biktarvy, Boceprevir, Bulevirtide, Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Edoxudine, Efavirenz, Elvitegravir, Emtricitabine, Enfuvirtide, Entecavir, Etravirine (Intelence), Famciclovir, Fomivirsen, Fosamprenavir, Foscarnet, Ganciclovir (Cytovene), Ibacitabine, Ibalizumab (Trogarzo), Idoxuridine, Imiquimod, Imunovir, Indinavir, Lamivudine, Letermovir (Prevymis), Lopinavir, Loviride, Maraviroc, Methisazone, Moroxydine, Nelfinavir, Nevirapine, Nexavir (formerly Kutapressin), Nitazoxanide, Norvir, Oseltamivir (Tamiflu), Penciclovir, Peramivir, Penciclovir, Peramivir (Rapivab), Pleconaril, Podophyllotoxin, Raltegravir, Remdesivir, Ribavirin, Rilpivirine (Edurant), Rilpivirine, Rimantadine, Ritonavir, Saquinavir, Simeprevir (Olysio), Sofosbuvir, Stavudine, Taribavirin (Viramidine), Telaprevir, Telbivudine (Tyzeka), Tenofovir alafenamide, Tenofovir disoproxil, Tenofovir, Tipranavir, Trifluridine, Trizivir, Tromantadine, Truvada, Umifenovirk, Valaciclovir, Valganciclovir (Valtrex), Vicriviroc, Vidarabine, Zalcitabine, Zanamivir (Relenza), Zidovudine, and combinations thereof. As used herein, the recitation of any anti-viral agent inherently encompasses the pharmaceutically acceptable salts thereof.

### Corticosteroids

Corticosteroids are well-known in the art. Corticosteroids mimic the effects of hormones that the body produces naturally in your adrenal glands. Corticosteroids can suppress inflammation and can reduce the signs and symptoms of inflammatory conditions (e.g., arthritis and asthma). Corticosteroids can also suppress the immune system. Corticosteroids can act on a number of different cells (e.g., mast cells, neutrophils, macrophages and lymphocytes) and a number of different mediators (e.g., histamine, leukotriene, and cytokine subtypes).

Steroids include, but are not limited to, the following: triamcinolone and its derivatives (e.g., diacetate, hexacetonide, and acetonide), betamethasone and its derivatives (e.g., dipropionate, benzoate, sodium phosphate, acetate, and valerate), dexamethasone and its derivatives (e.g., dipropionate and valerate), flunisolide, prednisone and its derivatives (e.g., acetate), prednisolone and its derivatives (e.g., acetate, sodium phosphate, and tebutate), methylprednisolone and its derivatives (e.g., acetate and sodium succinate), fluocinolone and its derivatives (e.g., acetonide), diflorasone and its derivatives (e.g., diacetate), halcinonide, desoximetasone (desoxymethasone), diflucortolone and its derivatives (e.g., valerate), flucloronide (fluclorolone acetonide), fluocinonide, fluocortolone, fluprednidene and its derivatives (e.g., acetate), flurandrenolide (flurandrenolone), clobetasol and its derivatives (e.g., propionate), clobetasone and its derivatives (e.g., butyrate), alclometasone, flumethasone and its derivatives (e.g., pivalate), fluocortolone and its derivatives (e.g., hexanoate), amcinonide, beclometasone and its derivatives (e.g., dipropionate), fluticasone and its derivatives (e.g., propionate), difluprednate, prednicarbate, flurandrenolide, mometasone, and desonide. As used herein, the recitation of a corticosteroid inherently encompasses the pharmaceutically acceptable salts thereof.

### Analgesics

The compositions of the present disclosure can also be used in combination therapies with opioids and other analgesics, including narcotic analgesics, Mu receptor antagonists, Kappa receptor antagonists, non-narcotic (i.e., non-addictive) analgesics, monoamine uptake inhibitors, adenosine regulating agents, cannabinoid derivatives, Substance P antagonists, neurokinin-1 receptor antagonists and sodium channel blockers, among others. Preferred combination therapies comprise a composition useful in methods described herein with one or more compounds selected from aceclofenac, acemetacin, .alpha.-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis (acetylsalicylate), amfenac, aminochlorthenoxazin, 3-amino-4-hydroxybutyric acid, 2-atnino-4-picoline, aminopropylon, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, .alpha.-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyalutninum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lomoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen and zomepirac. Analgesics are well known in the art. See, for example, The Merck Index, 12th Edition (1996), Therapeutic Category and Biological Activity Index, and the lists provided under “Analgesic”, “Anti-inflammatory” and “Antipyretic”. As used herein, the recitation of an analgesic inherently encompasses the pharmaceutically acceptable salts thereof.

### Cleaning Agents

Cleaning agents include hypoallergenic, non-irritating, disinfecting, antimicrobial, sterile solutions, for example gentle soap, saline solutions, alcohol solutions or gels, diluted vinegar solutions, wipes, etc., for, e.g., hand washing, wiping, scrubbing, and the like.

### Lubricants

Lubricants for use with vaginal molds may be, for example, water-based gels, water-based-liquids, aloe vera-infused, PH-balanced, silicone-based liquid, glycerin-free, unscented, hypoallergenic, non-staining, water and silicone hybrids, combinations thereof and the like.

### E. Kits

Disclosed herein are kits comprising one or more disclosed vaginal molds. In an aspect, a disclosed kit can comprise one or more agents or therapeutic agents. “Agents” and “Therapeutic Agents” are known to the art and are described supra. In an aspect, the one or more agents can treat, prevent, inhibit, and/or ameliorate one or more comorbidities in a subject. In an aspect, one or more active agents can treat, inhibit, prevent, and/or ameliorate a vaginal stenosis, and/or a vaginal stenosis related complication.

In an aspect, a disclosed kit can comprise at least two components constituting the kit. Together, the components constitute a functional unit for a given purpose (such as, for example, treating a subject diagnosed with or suspected of having vaginal stenosis). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed vaginal mold and a therapeutic agent, or a combination of therapeutic agents, and a label or package insert with instructions for use. In an aspect, suitable containers include, for example, bottles, vials, syringes, blister pack, etc. The containers can be formed from a variety of materials such as, cardboard, glass, or plastic.

The container can hold at least one disclosed vaginal mold, and at least one therapeutic agent or a combination thereof. The label or package insert can indicate that a disclosed one or more vaginal molds and one or more therapeutic agents or a combination thereof can be used for treating, preventing, inhibiting, and/or ameliorating vaginal stenosis or complications and/or symptoms associated with vaginal stenosis. A kit can comprise additional components necessary for administration such as, for example, other buffers, diluents, filters, and syringes.

In one aspect a kit comprises at least one disclosed vaginal mold further comprising at least one internal channel to allow for in situ administration of one or more therapeutic agent (e.g., vaginal estrogen cream, antibacterial/antifungal cream, anti-inflammatory/pain relief medication, vaginal moisturizer), and/or irrigation solution for cleaning or flushing with, for example, saline solutions.

In another aspect a kit comprises one or more disclosed vaginal molds of, for example, various sizes, and at least one therapeutic agent. In one aspect, a kit comprises disclosed vaginal molds of 3 incrementally larger sizes for gradual upsizing over time, at least one therapeutic agent, such as an antibiotic agent, pain relief medication, a moisturizer, and a lubricant to help introduce the vaginal mold. In an aspect, a kit can comprise a plurality of disclosed vaginal molds, at least one disclosed therapeutic agent, a disclosed lubricant to help with insertion, a cleaning agent for hand washing the devices, and user instructions.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

### INDUSTRIAL APPLICABILITY

The embodiments of the invention are further illustrated by the following non-limiting examples.

### Example 1

### 3-Dimensional Printed Vaginal Molds Were Successfully Used Following McIndoe Neovagina Surgery

Surgical vaginoplasty is a highly successful treatment for congenital absence of the vagina. One key to long-term success is the use of an appropriate vaginal mold in the immediate postoperative period. Congenital absence of the vagina and uterus are present in as many as 0.2-7% of females (Walters M D, et al. (2015) Urogynecology and Reconstructive Pelvic Surgery (4th ed.) Elsevier Inc). While many of these conditions are diagnosed around the time of puberty, the psychological impacts that accompany them can be life-long. Surgical vaginoplasty is the preferred method of treatment when vaginal dilation is not an option due to patient discomfort or preference or when previous dilator treatment has failed. The most performed vaginoplasty procedure is the McIndoe procedure. Traditionally, the McIndoe procedure involves creation of a neovaginal orifice followed by placement of a split thickness skin graft and insertion of a vaginal mold to maintain patency.

Overall, the procedure is seen as highly effective with up to 79% satisfaction rate (Klingele C J, et al. (2003) McIndoe procedure for vaginal agenesis: long-term outcome and effect on quality of life. Am J Obstet Gynecol. 189(6): 1569-1572). This reconstruction is not without risks, however, with complications reported in up to 10% of cases (Klingele et al. (2003)). Risks associated include fistula formation (1%), graft site infection (5.5%), and contractures (variable with mold compliance), and graft detachment (Walters et al. (2015)). The development of these complications is associated with a combination of factors including anatomic variances and patient compliance with mold placement.

One key to long term success of the McIndoe procedure is the use of an appropriate vaginal mold. An unsuitable mold could result in inappropriate drainage, detachment of the graft, fistulization, and loss of the surgically created space. A mold that is not conducive to patient self-application could lead to increased risk of contracture from insufficient use. A number of studies have been performed attempting to find the ideal mold, each with its own risks and benefits (Patnana A K, et al. (2019) Simple and novel technique for fabrication of prosthetic vaginal dilators. BMJ Case Rep. 12(4); Concannon M J, et al. (1993) An intraoperative stent for McIndoe vaginal construction. Plast Reconstr Surg. 91(2):367-368; Bates G W, et al. (1983) Management of vaginal agenesis: report of a case. J Miss State Med Assoc. 24(1):8-11; Han S E, et al. Experience with specially designed pored polyacetal mold dressing method used in McIndoe-style vaginoplasty. J Pediatr Urol.).

To design the mold, three-dimensional anatomical measurements of the planned neovaginal space were collected from volumetric computed tomography imaging. Using both these measurements and surgeon input, the mold design was created using a primary superior sphere, two cylinders (one larger main superior cylinder and a smaller inferior drain conduit cylinder), and a torus ring. Lofting was used to connect the main cylinder to the drain cylinder. Parallel oblique sketch planes from the tangent of the main cylinder were used to trim the anterior and posterior aspects of the mold. A 2 mm wall created through an internal hollow function and a 3 mm cylindrical egress hole was made at the axis of the superior sphere to facilitate drainage through the mold. Three different sizes, measuring total lengths of 92, 83, and 74 mm, respectively, were provided as options for use (FIG. 10). Additionally, a 2 mm plateau was created on the cephalad (adjacent to the neovaginal apex) portion of each of the three sizes to produce an additional three options for fitting. All design work was original and produced using Materialise 3-matic Medical v. 16.0 (Materialise, Leuven, Belgium).

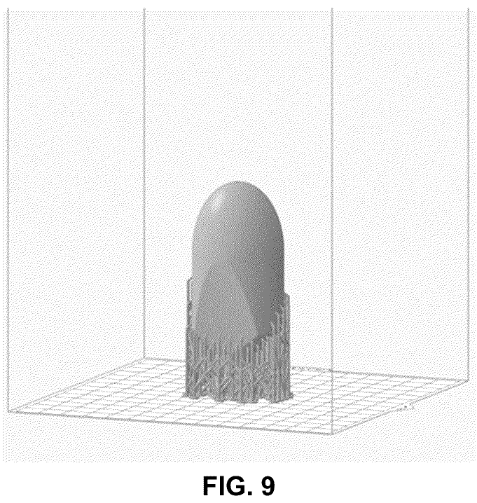

A commonly used dental resin (Denture Base, Formlabs, Boston, MA) was chosen for the material of the mold. This material was chosen given its long-standing safety profile in the oral cavity. Furthermore, dental resin is lightweight, nonporous, and non-abrasive. The molds were printed in duplicate on a Formlabs Form 3B vat photopolymerization 3D printer (Formlabs, Boston, MA). Referring to FIG. 9, the orientation and support of the structures was determined and performed by a member of the Anatomic Modeling Unit's Healthcare Technology Management staff to optimize 3D printing performance and part quality; internal supports were not feasible in this printing process due to the inability to remove them post-print, so the build setup was not trivial. In this optimized orientation, print time for one mold was approximately 9 hours.

When printing was complete, parts were removed from the build platforms and soaked in 99.0% Isopropyl Alcohol for 20 minutes. Supports were then carefully detached from the molds. At the juncture between mold and supports, a wet-sanding technique was used to smooth the surface. The sequence of sanding was as follows: 600 grit paper to sand the entire surface until layer lines were smooth, followed by 2500 grit paper until audible scratching no longer occurred, and finally a 3M micro-fine pad was used in a dry-sanding technique to remove any remaining tactile deformities on the surface of the mold (3M, St. Paul, MN; FIG. 2C). Various sized molds can be brought to the operating room and, once the vaginal topography was evaluated, a specific mold was sterilized prior to use per Formlabs Denture Base recommendations. See e.g., FIG. 10.

The use of the 3D printed vaginal mold in a 29-year-old woman who was born with cloacal exstrophy was studied. Patients with this condition have often had multiple surgeries to correct genitourinary, gastrointestinal, and orthopedic anomalies. Their pelvis takes a platypelloid-type shape with a reduced anterior-posterior diameter and nonunion of the symphysis; this topography precludes the use of standard vaginal molds. The anatomy of this patient was notable for a shortened anterior-posterior pelvic diameter, non-union of her symphysis pubis, prior bladder neck closure and creation of a neobladder and continent catheterizable channel, and anal atresia with the presence of an end colostomy. This patient had had two prior neovagina creation attempts but was unable to maintain patency. A repeat procedure was performed using a 3D printed mold that was custom made for her anatomy. She demonstrated ease with removal and maintenance of the 3D printed mold and, a year after her surgery, maintains a patent neovagina with a normal total vaginal length.

McIndoe procedural texts describe the use of a rigid solid cylinder mold for the first week following surgery, which was then replaced by a soft mold. Since its original documentation, many modifications of molds have been studied from material to shape.

As far as rigid molds, silicone dilators are firm and often more comfortable, however they have a tendency to collect debris on its rough surface. Acrylic, which is less giving in nature, is effective in maintaining the vaginal contour, however it applies more pressure to the vaginal walls and underlying organs, risking tissue necrosis and fistulization (Patnana A K, et al. (2019)). The idea of a soft mold was studied by Concannon et al who suggested filling a condom with a polyester fiber (Concannon M J, et al. (1993)). This mold could be compressed of all air prior to insertion and then “inflated” to accommodate the user's anatomy once in place (Concannon M J, et al. (1993)). While the technique was successful in all four cases studied, no mention was made of after cares and long-term outcomes (Concannon M J, et al. (1993)).

Wiser and Bates focused on shape when they created a polystyrene mold with a shortened and rounded base caudally to fit atop the perineal floor and anterior groove to accommodate the urethra (Bates G W, et al. (1983)). Hollow dilators have been suggested to aid in efflux of vaginal secretions along with decreased weight for patient comfort (Mohamed K, et al. (2021) Hollow prosthetic vaginal dilator—a novel technique. J Evolution Med Dent Sci 10(11):841-844).

The 3D printed mold used for the patient had a patient friendly knob on the distal end. This allowed for increased ease in placement and removal which, surprisingly, significantly increased compliance. Holes along the proximal and distal ends of the mold allowed for vaginal secretion egress, lowering the risk of infection, and contained seroma or hematoma. The narrowing at the area of the urethra helped to prevent obstructed urination, decreasing the risk of fistulization.

The use of the 3D printer for customizable vaginal molds opened the door not only to increased patient satisfaction, but also decreased complications. Significantly, these models can be customized to the unique anatomy of their users, as was the case with the patient in this study. This aspect will hold utmost significance in patients with a history of prior pelvic radiation, surgery, or other anatomic irregularities, as with this case. Overall, the 3D printed vaginal molds was more anatomic than the alternative commercially available models, and the patient reported the mold was comfortable and user friendly.

### Example 2

### Additional Clinical Studies

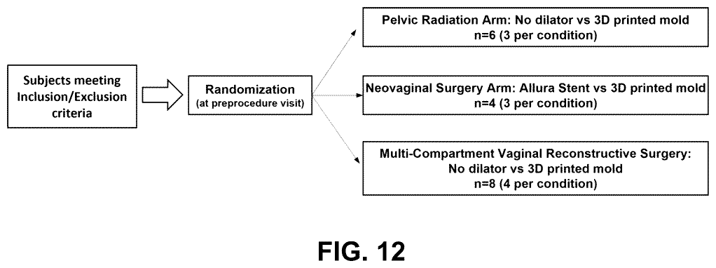

Subjects meeting criteria for inclusion in further testing can be found, as presented in flowchart 500 of FIG. 12, by targeting procedures such as neovaginal surgeries, pelvic radiation, and multi-compartment (anterior, posterior, and apical vagina) vaginal reconstructive surgery, all of which carry a risk of vaginal stricture and loss of length. By targeting these specific procedures, a notable event is established, vaginal topography is measured, and the subjects report outcomes before and after the notable event. The subjects are randomized to the three study arms: The comparators in the arms represent use of the 3D printed mold versus standard of care. Currently, dilator therapy for women undergoing pelvic radiation is not prophylactically prescribed, despite the 40% genitourinary toxicity following XRT. Using a stent or mold following neovaginal surgery is standard of care; therefore, it is possible to compare the commercially available stent to the embodiments of vaginal molds disclosed in this application. Finally, the multi-compartment vaginal reconstruction group is a group undergoing transvaginal surgery in all 3 compartments (anterior, posterior, apical) of the vagina. While they are at risk for stricture and agglutination of suture lines, standard of care does not involve postoperative dilator/mold use. A comparison can be made of no dilator/mold versus the 3D printed device for this group. The sample size represents a typical sample for a Phase I trial as well as convenience sample for an 18-month trial.

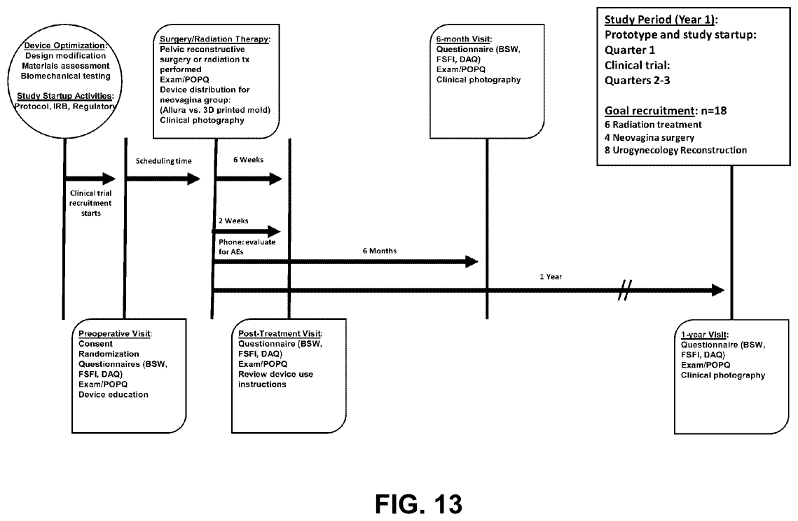

As shown in more detail on the workflow of FIG. 13, the subjects receive care based on their randomization within their study arm. All subjects undergo an adverse event (“AE”) phone call 2 weeks after their intervention. Severe is immediately reported to the Data and Safety Monitoring Board. Non-Severe AEs are collected and later analyzed by use or non-use of the 3D printed mold. Subjects have a standard 6-week post-intervention visit, along with a 6-month, and 1-year visit. The primary outcome is change in total vaginal length from the time of intervention to 1-year after intervention. Secondary outcomes include differences in patient reported outcome and differences in reported AE.

The preceding examples can be repeated with similar success by substituting the generically or specifically described operating conditions or parameters of this invention for those used in the preceding examples.

What is claimed is:

1. A vaginal mold comprising:
a rigid, at least partially hollow body defining a cavity and comprising a proximal end, a middle section, and a distal end;
wherein the proximal end comprises a spherical dome-like, bulbous, globular shape;
the middle section comprises tapering right and left sides; and
the distal end comprises front and back tapering sides and bottlenecks to form a neck that ends into a knob comprising an opening to the cavity.

2. The vaginal mold of claim 1, further comprising one or more perforations to facilitate drainage of vaginal fluids through the opening of the cavity.

3. The vaginal mold of claim 2, wherein the one or more perforation are disposed on the proximal end of the body.

4. The vaginal mold of claim 1, wherein the proximal end has a rotationally symmetric spherical shape.

5. The vaginal mold of claim 1, further comprising a superior plateau aspect.

6. The vaginal mold of claim 1, wherein the knob further comprises a lip.

7. The vaginal mold of claim 1, wherein the body is made of biocompatible plastic.

8. The vaginal mold of claim 1, further comprising one of more internal channels to facilitate flow of fluid to the exterior surface of the mold that contacts a patient's vaginal tissue.

9. A kit comprising a plurality of the vaginal molds of claim 1, wherein the molds are provided in incrementally larger sizes for gradual upsizing over time.

10. The kit of claim 7, wherein the plurality comprises 2 or more vaginal molds.

11. The kit of claim 8, wherein the plurality comprises 3 or more vaginal molds and the kit further comprises at least one therapeutic agent, and a package insert with instructions for use.

12. A method of treating vaginal stenosis in a subject in need thereof using the vaginal mold of claim 1, the method comprising: inserting the vaginal mold for a specified period of time to follow a treatment regimen.

13. The method of claim 12 further comprising administering to the subject an effective amount of one or more therapeutic agents.

14. The method of claim 13 wherein vaginal mold can be inserted and removed with ease by the patient, and the regimen indicates gradual upsizing of incrementally larger vaginal mold sizes over time.

15. A method of fabricating the vaginal mold of claim 1, the method comprising:
obtaining one or more patient pelvic measurements;
customizing the vaginal mold for the patient by generating a CAD 3D model of the vaginal mold from the patient measurements; and
3D printing the vaginal mold.

16. The method of claim 15, further comprising post-processing the 3D printed vaginal mold.

17. The method of claim 15, wherein obtaining one or more patient pelvic measurements comprises using imaging modalities.

18. The method of claim 15, wherein the generating a CAD 3D model comprises using one or more saved compatible templates.

19. The method of claim 16 wherein the post-processing the 3D printed vaginal mold comprises smoothing the outer surface of the vaginal mold.

20. A vaginal mold, comprising:
a rigid, at least partially hollow body defining a cavity and comprising a proximal end, a middle section, and a distal end;
wherein the proximal end comprises a spherical rotationally symmetric, dome-like, bulbous, globular shape comprising one or more drainage perforations;
wherein the middle section comprises tapering right and left sides; and
wherein the distal end comprises front and back tapering sides and bottlenecks to form a neck that ends into a knob comprising a lip and an opening to the cavity.
